(19) United States
(12) Patent Application Publication
(10) Pub. No.: US 2020/0383323 A1
(43) Pub. Date: Dec. 10, 2020

(54) ELECTRONIC DEVICE SCREEN COVERS AND ELECTRONIC DEVICES WITH BIOACTIVE MATERIALS

(71) Applicants: Mark Sisson, Forest, VA (US); Dennis Hackemeyer, Forest, VA (US)

(72) Inventors: Mark Sisson, Forest, VA (US); Dennis Hackemeyer, Forest, VA (US)

(21) Appl. No.: 16/878,996

(22) Filed: May 20, 2020

Related U.S. Application Data
(60) Provisional application No. 62/850,782, filed on May 21, 2019.
(60) Provisional application No. 63/000,359, filed on Mar. 26, 2020.

(51) Int. Cl. A01N 25/10; A61B 50/00; A01N 59/00; A01N 59/16; A01N 59/20; C09J 7/25; C09J 9/00; C09J 11/04; B32B 7/12; B32B 27/40; B32B 27/32; B32B 27/08; B32B 27/20; G06F 1/16

(52) U.S. Cl. CPC A01N 25/10; H04B 1/3827; A01N 59/00; A01N 59/16; A01N 59/20; C09J 7/25; C09J 9/00; C09J 11/04; B32B 7/12; B32B 27/40; B32B 27/32; B32B 27/08; B32B 27/20; G06F 1/1637; A61B 2050/002; A61B 2050/0065; C09J 2475/006; C09J 2301/16; B32B 2307/412; B32B 2457/208; B32B 2255/10; A61B 50/00

(57) ABSTRACT

Certain embodiments described herein are directed to electronic device screen covers that include one more bioactive materials. In some embodiments, the electronic device screen covers can be used with capacitive touch screens on electronic devices such as cellular phones, tablets, laptops, kiosk displays or other electronic devices that include screens. Methods of using the screen covers and screens to reduce spread of infections are also described.

### PRIORITY APPLICATIONS

This application is related to and claims priority to and the benefit of U.S. Application No. 62/850,782 filed on May 21, 2019 and U.S. Application No. 63/000,359 filed on Mar. 26, 2020, the entire disclosure of each of which is hereby incorporated herein by reference for all purposes.

### TECHNOLOGICAL FIELD

Certain embodiments described herein are related to electronic device screen covers. More particularly, certain configurations are directed to electronic device screen covers that include one or more bioactive materials.

### BACKGROUND

Electronic devices often include capacitive touch screens which permit an end user to interact with the device. A user can select and use application software using, for example, a stylus or their fingers.

### SUMMARY

Certain aspects described herein are directed to electronic screen covers that comprise one or more bioactive materials. For example, the screen cover may comprise a bioactive material that can kill and/or inactivate bioorganisms that are transferred from a user to the screen cover. Electronic device screens with integral screen covers and/or surface coatings are also described. In some instances, the adhesive layer used to couple the screen cover to the underlying screen can be omitted, and the screen can be held in place by way of static cling, through wetting or hydrophobic interactions or through means other than an adhesive. The screen covers may comprise a single cover or be configured as tear offs to expose an underlying new screen cover as desired.

In an aspect, an electronic device screen cover configured to couple to an electronic device screen comprises a release liner, a first adhesive layer coupled to the release liner, a substrate coupled to the first adhesive layer, a second adhesive layer coupled to the substrate, a carrier support material coupled to the second adhesive layer, and a surface coating coupled to the carrier support material, the surface coating comprising a bioactive material to inactivate or kill bioorganisms that contact the surface coating.

In certain embodiments, the bioactive material comprises at least one of titanium, silver, copper and zinc. In some examples, the carrier support material comprises a polyurethane. In some examples, the bioactive material comprises a photocatalyst comprising titanium, and wherein the bioactive material comprises at least one additional transition metal. In some embodiments, a second bioactive material can be embedded in the polyurethane of the carrier support material. In other examples, the bioactive material and the second bioactive material comprise different transition metals. In additional examples, the first adhesive layer comprises a residue free adhesive. In other examples, the carrier support material comprises a substantially planar shape. In certain embodiments, the electronic device screen cover is optically transparent. In other embodiments, the substrate comprises a polyolefin.

In an additional aspect, an electronic device screen cover configured to couple to an electronic device scree comprises a release liner, a first adhesive layer coupled to the release liner, a substrate coupled to the first adhesive layer, a second adhesive layer coupled to the substrate, and a carrier support material coupled to the second adhesive layer, wherein the carrier support material comprises an embedded bioactive material to inactivate or kill bioorganisms that contact the carrier support material.

In some configurations, the bioactive material comprises at least one of titanium, silver, copper and zinc. In other configurations, the carrier support material comprises a polyurethane. In certain embodiments, the bioactive material comprises a photocatalyst comprising titanium, and wherein the bioactive material comprises at least one additional transition metal. In other embodiments, a second bioactive material can be embedded in the polyurethane of the carrier support material. In some examples, the bioactive material and the second bioactive material comprise different transition metals. In certain examples, the first adhesive layer comprises a residue free adhesive. In other examples, the carrier support material comprises a substantially planar shape. In some examples, the electronic device screen cover is optically transparent. In other embodiments, the substrate comprises a polyolefin.

In another aspect, an electronic device comprises an electronic device screen cover that comprises a first adhesive layer coupled to the screen, a substrate coupled to the first adhesive layer, a second adhesive layer coupled to the substrate, a carrier support material coupled to the second adhesive layer, and a surface coating coupled to the carrier support material, the surface coating comprising a bioactive material to inactivate or kill bioorganisms that contact the surface coating.

In some examples, the bioactive material comprises at least one of titanium, silver, copper and zinc. In other examples, the carrier support material comprises a polyurethane. In some embodiments, the bioactive material comprises a photocatalyst comprising titanium, and wherein the bioactive material comprises at least one additional transition metal. In other examples, a second bioactive material can be embedded in the polyurethane of the carrier support material. In other embodiments, the bioactive material and the second bioactive material comprise different transition metals. In certain examples, the first adhesive layer comprises a residue free adhesive. In other examples, the carrier support material comprises a substantially planar shape. In some embodiments, the electronic device screen cover is optically transparent. In additional examples, the substrate comprises a polyolefin.

In another aspect, an electronic device comprises an electronic device screen cover that comprises a first adhesive layer coupled to the screen, a substrate coupled to the first adhesive layer, a second adhesive layer coupled to the substrate, and a carrier support material coupled to the second adhesive layer, wherein the carrier support material comprises an embedded bioactive material to inactivate or kill bioorganisms that contact the carrier support material.

In certain examples, the bioactive material comprises at least one of titanium, silver, copper and zinc. In other examples, the carrier support material comprises a polyurethane. In some embodiments, the bioactive material comprises a photocatalyst comprising titanium, and wherein the bioactive material comprises at least one additional transition metal. In further examples, a second bioactive material can be embedded in the polyurethane of the carrier support material. In other examples, the bioactive material and the second bioactive material comprise different transition metals. In certain embodiments, the first adhesive layer comprises a residue free adhesive. In other embodiments, the carrier support material comprises a substantially planar shape. In some examples, the electronic device screen cover is optically transparent. In other examples, the substrate comprises a polyolefin.

In an additional aspect, an electronic device comprises an integral electronic device screen cover that comprises a substrate coupled to the screen, a first adhesive layer coupled to the substrate, a carrier support material coupled to the second adhesive layer, and a surface coating coupled to the carrier support material, the surface coating comprising a bioactive material to inactivate or kill bioorganisms that contact the surface coating.

In certain examples, the carrier support material comprises a substantially planar shape. In other examples, the electronic device screen cover is optically transparent. In some embodiments, the substrate comprises a polyolefin and the carrier support material comprises a polyurethane. In other embodiments, the bioactive material comprises at least one of titanium, silver, copper and zinc.

In an additional aspect, an electronic device comprises an integral electronic device screen cover that comprises a substrate coupled to the screen, a first adhesive layer coupled to the substrate, and a carrier support material coupled to the first adhesive layer, wherein the carrier support material comprises an embedded bioactive material to inactivate or kill bioorganisms that contact the carrier support material.

In certain embodiments, the carrier support material comprises a substantially planar shape. In some examples, the integral electronic device screen cover is optically transparent. In other examples, the substrate comprises a polyolefin and the carrier support material comprises a polyurethane. In some instances, the bioactive material comprises at least one of titanium, silver, copper and zinc.

In another aspect, a mobile device comprises a display screen and a mobile device screen cover coupled to the display screen, wherein the mobile device screen cover comprises a substrate coupled to the display screen, a first adhesive layer coupled to the substrate, and a surface coating coupled to the first adhesive layer, wherein the surface coating comprises a bioactive material to inactivate or kill bioorganisms that contact the carrier support material.

In another aspect, a mobile device comprises a display screen and a mobile device screen cover coupled to the display screen, wherein the mobile device screen cover comprises a substrate coupled to the display screen, a first adhesive layer coupled to the substrate, and a carrier support material coupled to the first adhesive layer, wherein the carrier support material comprises an embedded bioactive material to inactivate or kill bioorganisms that contact the carrier support material.

In an additional aspect, a mobile device comprises a display screen and a mobile device screen cover coupled to the display screen, wherein the mobile device screen cover comprises a first adhesive layer coupled to the display screen, a substrate coupled to the first adhesive layer, a second adhesive layer coupled to the substrate, a carrier support material coupled to the second adhesive layer, and a surface coating coupled to the carrier support material, the surface coating comprising a bioactive material to inactivate or kill bioorganisms that contact the surface coating.

In another aspect, a mobile device comprises a display screen and a mobile device screen cover coupled to the display screen, wherein the mobile device screen cover comprises a first adhesive layer coupled to the display screen, a substrate coupled to the first adhesive layer, a second adhesive layer coupled to the substrate, and a carrier support material coupled to the second adhesive layer, wherein the carrier support material comprises an embedded bioactive material to inactivate or kill bioorganisms that contact the carrier support material.

In another aspect, a mobile device comprises a display screen, wherein the display screen comprises a surface coating comprising a bioactive material to inactivate or kill bioorganisms that contact the surface coating.

In an additional aspect, a mobile device comprises a display screen, wherein the display screen comprises a carrier support material and a surface coating coupled to the carrier support material, the surface coating comprising a bioactive material to inactivate or kill bioorganisms that contact the surface coating.

In another aspect, a method comprises placing one or more of the screen covers or screens described herein onto another article or device to facilitate transfer of infectious organisms, infectious virus, infectious viral agents or infectious viral particles from a human to the placed screen cover or screen. In some examples, the placed screen cover or screen comprises a bioactive material to kill or inactivate the transferred infectious organisms, infectious virus, infectious viral agents or infectious viral particles. In certain embodiments, the virus that is transferred and inactivated is a coronavirus.

In an additional aspect, a method comprises reducing community spread of an infection by placing one or more of screen covers or screens described herein onto another article or device to facilitate transfer of infectious organisms, infectious virus, infectious viral agents or infectious viral particles from a user to the placed screen cover or screen. In some examples, the placed screen cover or screen comprises a bioactive material to kill or inactivate the transferred infectious organisms, infectious virus, infectious viral agents or infectious viral particles so successive humans touching the placed screen cover or screen do not become infected by the infectious organisms, infectious virus, infectious viral agents or infectious viral particles. In certain embodiments, the virus that is transferred and inactivated is a coronavirus.

In another aspect, a method of treating a human infected with an infectious organism, an infectious virus, an infectious viral agent or an infectious viral particle comprises administering to the infected human a therapeutic to treat the infection, and reducing spread of the infection from the infected human to third parties by placing one or more of the screen covers or screens described herein onto another article or device facilitate transfer of the infectious organisms, infectious virus, infectious viral agents or infectious viral particles from the infected human to the placed screen cover or screen. In certain examples, the placed screen cover or screen comprises a bioactive material to kill or inactivate the transferred infectious organisms, infectious virus, infectious viral agents or infectious viral particles so successive humans touching the placed screen cover or screen do not become infected by the infectious organisms, infectious virus, infectious viral agents or infectious viral particles. In some embodiments, the therapeutic is an antimicrobial agent or an antiviral agent or comprises an antimicrobial agent and an antiviral agent

In an additional aspect, a kit comprises a therapeutic to treat a human infected with an infectious organism, an infectious virus, an infectious viral agent or an infectious viral particle, and a screen cover or screen comprising a bioactive material to kill or inactivate any infectious organisms, infectious virus, infectious viral agents or infectious viral particles transferred from the infected human so successive humans touching the placed screen cover or screen do not become infected by the infectious organisms, infectious virus, infectious viral agents or infectious viral particles. In certain embodiments, the therapeutic of the kit is an antimicrobial agent or an antiviral agent or comprises an antimicrobial agent and an antiviral agent. In some examples, the kit comprises written or electronic instructions for using the therapeutic to treat the infection and using the screen cover or screen to prevent or reduce spread of the infection.

Additional aspects, examples, configurations and embodiments are described in more detail below.

### BRIEF DESCRIPTION OF THE SEVERAL VIEWS OF THE DRAWINGS

FIG. 1 is an illustration showing an electronic screen cover placed on an electronic device screen, in accordance with some examples;

FIG. 2 is an illustration showing a bioactive material present in a surface coating, in accordance with some examples;

FIG. 3 is an illustration showing a bioactive material embedded in a carrier support material of a surface coating, in accordance with some examples;

FIG. 4 is an illustration showing a bioactive material present in a surface coating and embedded in a carrier support material, in accordance with some examples;

FIGS. 5A, 5B, 5C, 5D and 5E show shapes for different carrier support materials;

FIG. 6 is an illustration of a mobile device comprising a mobile device screen cover, in accordance with some examples;

FIG. 7 is a side view of a display screen comprising a surface coating, in accordance with some embodiments; and

FIG. 8 is a table showing reduction in coronavirus using an article including a bioactive material; and

FIG. 9 is a table showing reduction in E. coli using an article including a bioactive material.

### DETAILED DESCRIPTION

Certain articles are described below in connection with electronic devices and electronic device covers. The electronic device may take many different forms including a cellular phone, laptop, tablet, display screen, kiosk screen or other electronic devices that include a microprocessor and a display screen. As noted herein, the electronic device screen cover is typically configured as a screen cover that can couple to a front surface of a display of an electronic device. In some instances, the screen cover may be integral to the screen, whereas in other examples, the screen cover may be coupled to the screen through an adhesive layer present on the screen cover. The screen cover is typically non-electronic and does not comprise any moving parts but does include one or more bioactive materials as noted in more detail below.

Electronic device screen covers are described in certain instances as comprising a bioactive material that can kill or inactivate bioorganisms. The term “bioorganism” is intended to include, but is not limited to, bacteria, fungi and bacterial and fungal spores as well as any viruses or portions thereof, e.g., any membrane components or other components of the bacteria, fungi or virus that may be secreted. Illustrative bioorganisms that are targeted include gram positive and gram negative bacteria, Staphylococcus, Escherichia coli, Propionibacteria, Corynebacteria, dermobacteria, and micrococci, Tinea, Candida, flu virus, adenoviruses and other bacterial, fungi and viruses. The bioactive material may also be effective to inactivate or render non-toxic secreted proteins and materials such as endotoxins or other toxins.

In certain embodiments, the articles described herein may comprise one or more surface coatings or layers. In some examples, the surface coating may comprise a bioactive material on an outer surface of the surface coating. In other instances, the surface coating may comprise an embedded bioactive material. In additional examples, the surface coating may comprise a bioactive material on an outer surface of the surface coating and may also comprise an embedded bioactive material. For example, as the bioactive material on the outer surface breaks down or is otherwise removed by contact, the embedded bioactive material may still be present to kill or inactivate bioorganisms. In some examples, the surface coating or layer may comprise a carrier material or support that can receive the bioactive material on its surface or can permit embedding of the bioactive material. While not wishing to be bound by any one configuration, the surface coating can be a non-transfer surface coating such that no or little material is transferred to a user contacting the surface coating with their hands, fingers or other body part. For example, bioorganisms can be transferred from the user to the surface coating where they are inactivated, killed or oxidized by the bioactive material.

In certain embodiments, the bioactive material in the surface coating may be photoactivatable and/or photorechargeable to permit continuous use and reuse of the article. For example, the surface coating can be exposed to infrared, visible, ultraviolet or light of other wavelengths to activate the bioactive material in the surface coating such that the bioactive material can function as a photocatalyst. For example, the bioactive material may comprise photocatalytic titanium dioxide or other photocatalytic transition metal materials. Once activated, the bioactive material can, for example, oxidize groups or constituents on bioorganisms to inactivate and/or kill them. The bioactive material may be photorechargeable by exposing the bioactive material to additional light for an activation period, e.g., 10 second or more, 20 seconds or more, 30 second or more, 1 minute or more, etc. Reactivation recharges the bioactive material for addition use. While the bioactive material may be exposed to light for a suitable period, actual recharging of the material can occur quickly, e.g., within a few microseconds, milliseconds, etc.

In some configurations, the bioactive material may comprise a metal or a material which can release ions, e.g., within the carrier support. For example, the bioactive material can be a transition metal or a transition metal containing material that includes one or more transition metals which can be present in ionic form and/or complexed with one or more ligands. Without wishing to be bound by any one configuration, the transition metal may be present in different forms in the carrier material including free ions and complexed ions. In some examples, the transition metal may be any one or more of scandium, titanium, vanadium, chromium, manganese, iron, cobalt, nickel, copper, zinc, yttrium, zirconium, niobium, molybdenum, technetium, ruthenium, rhodium, palladium, silver, cadmium, hafnium, tantalum, tungsten, rhenium, osmium, iridium, platinum, gold, and mercury, with non-radioactive transition materials being desirable to use and with ionized forms of the transition metals being desirable for use in some instances. In some instances, the bioactive material may comprise two or more different transition metals each of which can independently be present as free ions or complexed with a ligand or other groups. In other instances, the bioactive material may comprise three or more different transition metals each of which can independently be present as free ions or complexed with a ligand or other groups. Where the bioactive material is a photocatalyst, the bioactive material may comprise one or more transition metals. Where the bioactive material is embedded in a surface coating or a carrier support material, the bioactive material desirably can release transition metal ions which can bind to and/or be taken up by the bioorganisms.

In certain embodiments, the transition metal material can be selected to oxidize constituents or groups present on the bioorganisms to kill or inactivate the bioorganisms. For example, the transition metal or transition metal material may function as a photocatalyst and can be activated by exposure of the surface coating to ultraviolet or visible light. Subsequent to initial use, the transition metal or transition metal material can be photo-recharged by exposing the surface coating to additional ultraviolet or visible light. The exact interval where at least 50% of the material remains in an activated form may vary from a few days to a few weeks or even a few months. At any time, a certain amount of the bioactive material may be present in an active state to photocatalyze the received bioorganisms while some portion of the bioactive material may be present in an inactive state. Recharging may be performed, for example, when the amount of bioactive material in the active state drops below a certain percentage, e.g., 50%, 40%, 30%, 20% or even 10%.

In certain configurations, the bioactive material can also be present on top of the carrier support material as a separate surface coating. For example, the support material may comprise embedded bioactive material and additional bioactive material may be present as a separate surface layer or surface coating on top of the carrier support material comprising the embedded bioactive material. In some cases, bioactive material can migrate from the carrier support material into the outer surface coating or layer to replenish the bioactive material as it is consumed or leaches off. In other instances, the embedded bioactive material does not migrate but can remain active within the carrier support material to kill and/or inactivate bioorganisms. Where a surface coating of bioactive material is deposited on top of a layer comprising the embedded bioactive material in the carrier support material, the surface coating may comprise a transition metal or a transition metal containing material that includes one or more transition metals which can be present in ionic form and/or complexed with one or more ligands. The transition metal bioactive material deposited on top of the carrier support material may be present in different forms in the carrier material including free ions and complexed ions. In some examples, the transition metal present on top of a carrier support material may comprise one or more of scandium, titanium, vanadium, chromium, manganese, iron, cobalt, nickel, copper, zinc, yttrium, zirconium, niobium, molybdenum, technetium, ruthenium, rhodium, palladium, silver, cadmium, hafnium, tantalum, tungsten, rhenium, osmium, iridium, platinum, gold, and mercury, with non-radioactive transition material being desirable to use and with ionized forms of the transition metals being desirable for use in some instances. In some instances, the transition metal bioactive material deposited on top of the carrier support material may comprise two or more different transition metals each of which can independently be present as free ions or complexed with a ligand or other groups. In other instances, the transition metal material deposited on top of the carrier support material may comprise three or more different transition metals each of which can independently be present as free ions or complexed with a ligand or other groups.

In certain examples, the carrier support material of the articles described herein typically is selected to be able to withstand physical contact of the screen covers. For example, the carrier material may be a polymeric material that can be disposed on a substrate in a desired shape and using suitable methods, e.g., printing, spraying, coating, dip coating, rolling or using other methods. As noted herein, a bioactive material of the surface coating can be present on or in the carrier material (or both) and used to inactivate or kill bioorganisms. In some embodiments, the carrier support material can be selected such that it retain the bioorganisms within the surface coating, e.g., prevents transfer of the bioorganisms back to a second user contacting the articles. In some embodiments, the carrier material may comprise one or more thermoplastics or one or more thermosetting materials. For example, polyurethanes, polyacrylates, and copolymers comprising polyurethanes, polyacrylates or other polymeric materials that are optically transparent when placed on a substrate can be used. In other instances, the carrier support material may be a polyester, an epoxy resin, a polyimide, a silicone resin, a vinyl ester resin, a polycarbonate, a polyetherimide, a polypropylene, a polyphenylene oxide, a polyphenylene sulfide or other resins or materials that are desirably optically transparent, though the carrier support materials may be opaque or partially opaque if desired.

In certain embodiments, the surface coating may be hard, soft, non-compressible or compressible depending on the end use and configuration of the final article. In some examples, the carrier support material may be elastic and optionally comprise one or more elastomeric materials. For example, upon depression of the article by a user, the article may spring back to an initial position after removal of the depressing force. In some instances, rubber, natural rubber, synthetic rubber or other rubber based materials may be present in the carrier support material.

In other instances, the bioactive material may form clusters on top of the carrier support material with open space present between the clusters. In such instances, it may be desirable to include embedded bioactive material in the carrier support material in case bioorganisms do not contact any of the surface clusters of the bioactive material.

In certain embodiments, the articles described herein are typically placed on top of another article or device and can be designed to permit viewing of the underlying article or device. For example, the entire article may be produced using materials which are generally transparent, e.g., over visible wavelength ranges, such that viewing of the underlying article or device is permitted. The article need not transmit 100% of the light but is generally transparent enough so underlying text or other features of the device or article is viewable using the naked eye. Even though the articles can be optically transparent, they may be colored if desired. Alternatively, the articles may be colorless. In addition, the articles generally do not interfere with operation of the underlying device.

In certain embodiments, the layers of the articles may comprise fibers, elastomers or other materials to alter the overall properties of articles. For example, elastomeric fibers may be present to permit depression or compression of the articles during use. Further, additional materials may be present to provide touch indicia such as Braille, raised letters or numbers or other features.

In certain configurations, the articles described herein generally comprise a substrate upon which the surface coating (or other layers) is placed. In certain embodiments, suitable substrates that can be used with the surface coating described herein may be optically transparent, printed or may be opaque if desired. In certain examples, the exact material used in the substrate can vary depending on the intended use environment of the article. In some examples, the substrate may comprise a paper, a fabric, a metal, a non-metal, a plastic, a ceramic, a glass, a fiberglass, a stone, a wood, a rubber, a foam, a textile, cardboard, a vinyl material, concrete, asphalt, leather, suede, a polymeric material or other materials. In embodiments where papers are used, the paper may be acid-free or may be designed to be present in its use environment for a desired period without substantial degradation. In examples where a fabric is used, the fabric may be a woven fabric, a non-woven fabric, a polyester fabric such as, for example, a draw textured yarn (DTY) polyester fabric, a polyester-copolymer fabric and other fabrics commonly used to receive inks and colorants using printing techniques, lithographic techniques or other techniques. For example, polyester DTYs are effective to absorb and retain inks and other colorants. In addition, polyester DTYs can permit even distribution of the inks or colorants to provide desirable indicia. Illustrative DTY' s can be found, for example in U.S. Patent Publication No. 20110008563 filed on Jul. 9, 2009, the entire disclosure of which is incorporated herein by reference. Where the substrate is a ceramic, the ceramic may be, for example, aluminum oxide, yttrium oxide, cerium oxide, beryllia, zirconia, a carbide, a boride, a nitride, a silicide or other ceramic materials. Where the substrate is a glass, the glass may be colored, non-colored, opaque, transparent or may include variable areas having different properties. If desired, the glass may include reinforcing fibers or other materials to strengthen the physical properties of the glass. Where the substrate is a stone, wood, rubber, foam or other material, the material may be porous such that physical indicia can be imparted to the material. If the material is highly porous, then it may be desirable to reduce the porosity of the material by first disposing an agent on the material that can occupy some of the pores of the material. In some embodiments, the substrate may be a plastic material such as, for example, a thermoplastic material or a thermosetting material.

In some examples, the substrate may comprise a polyolefin material that is optically transparent. For example, the substrate material may comprise a polyethylene, a polyethylene copolymer, a polyvinyl chloride, a polyvinyl chloride copolymer, a polypropylene or other polyolefins that are optically transparent at least to some degree. In some embodiments, the polyolefin may be non-porous and be configured to retain the surface coating and optionally other materials on a surface of the substrate. For example, while the surface coating may include some porosity to permit bioorganisms to penetrate into the surface coating, the substrate generally is non-porous or fully consolidated such that surface coating materials do not penetrate into the substrate. Illustrative substrates are commercially available from many different sources including, but not limited to, those from the PhotoTex® Group Inc. (Boardman, Ohio), Fusion Digital (Washington, Utah), Yupo (Chesapeake, Va.), Granwell (West Caldwell, N.J.), Superior Fabrics (Pompano Beach, Fla.), Worthen Industries (Nashua, N.H.) and other commercial sources. In some instances, the substrate can be flexible, rigid, semi-rigid, compressible or may have other physical properties as desired.

In some examples, the overall shape and thickness of the various layers may vary as desired and depending on the intended use of the article. In some examples, the carrier support material layer may comprise a thickness, for example, of about 0.5 mm to about 5 mm. Where a surface coating of bioactive material is present on top of the carrier support material layer, the surface coating thickness may be, for example, about 0.1 mm to about 1 mm. The overall thickness of the substrate can vary from about 0.1 mm to about 5 mm. The width and length of the articles depend on the end use of the articles and illustrative values are discussed below. The thickness of the bioactive material can be as little as 1-2 crystals, e.g., 7-10 nm or less.

In certain embodiments, one or more protective layers, materials or coatings may be present on the articles described herein. The protective layer, material or coating may be present between two or more other components of the articles as desired or within any one or more layers. In some examples, the protective layer, material or coating may be present on top of the active surface layer or coating. For example, in applications where the articles are used outside, a UV protective material, color fast material or other materials may be present on top of the active surface coating or mixed with it to protect it. The protective coating, material or layer can be porous to permit bioorganisms to be transferred from a user's hand or other body part to the underlying active surface coating or layer for inactivation, oxidation and/or killing. In other examples, the protective layer or material may be present on top of the substrate and used to render the substrate color fast or protect any ink or other colorant on the substrate from photobleaching, UV degradation or degradation due to other means. Suitable materials for use as a protective layer or coating include, but are not limited to, acrylates such as, for example, trimethylpropaneacrylate, epoxyacrylate, urethaneacrylate and other acrylates. Other polymeric materials including polyolefins, nanoparticles and the like may also be present as protective coatings.

In certain embodiments, the articles described herein typically comprise an adhesive layer between the substrate and a release liner. The adhesive layer can be designed to adhere the article to an underlying screen. In some examples, the adhesive can be a residue free adhesive such that removal of the article from the underlying device or article does not leave behind any adhesive on the surface of the screen. Illustrative adhesives include, but are not limited to, thermoplastic adhesives and thermosetting adhesives. For example, the adhesive may comprise one or more of adhesives or residue-free adhesives that are commercially available from 3M, Henkel, Shell Chemical Company, Kuraray Company and other commercial suppliers of adhesives. In some examples, the adhesive may comprise rubber or other elastomer and be a residue-free adhesive. In other examples, the adhesive can be a silicon based adhesive such as, for example, an organopolysiloxane adhesive. In some examples, the adhesive can include one or more cross-linkable groups such as, for example, an isocyanate group, an unsaturated hydrocarbon group, a sulfo group, a sulfhydryl group, an alkoxy group, a hydroxyl group, and other groups that can be cross-linked. In some embodiments, the adhesive can be used in combination with a crosslinking agent to facilitate cross-linking and/or curing of the adhesive. In some embodiments, the adhesive can include one or more materials, polymers or copolymers including, but not limited to, styrene block polymers such as, for example, a styrene and styrene/diene copolymer (SBS, SIS, SBR), a styrene/ethylene/butylene copolymer (SEBS) or a styrene/ethylene/propylene/styrene copolymers (SEPS), acrylate copolymers, a polyester urethane copolymer, an ethylene acrylate copolymer, a butyl rubber copolymer; a natural rubber copolymer; an ethylene/propylene copolymer; an ethylene/vinyl acetate copolymers, EPDM/PP, NR/PP, EVA/PVDC and NBR/PP, polyurethanes, polyether esters and polyether amides based copolymers or materials. Additional materials and groups can also be used to prepare the adhesive including, but not limited to, homo- or copolymers of 1,3-butadiene, 2-methyl-1,3-butadiene (isoprene), 1,3-pentadiene, 2-ethyl-1,3-butadiene, 2-propyl- 1,3-butadiene, 2-isopropyl-1,3-butadiene, 2-hexyl-1,3-butadiene, 2,3-dimethyl-1,3-butadiene, 2,3-diethyl-1,3-butadiene, 2-methyl-1,3-pentadiene, 2-methyl-1,3-hexadiene, 2-methyl-1,3-octadiene, 2-methyl-1,3-decadiene, 2,3-dimethyl-1,3-pentadiene, 2,3-dimethyl-1,3-hexadiene, 2,3-dimethyl-1,3-octadiene and 2,3-dimethyl-1,3-decadiene, 2-methyl-1,3-cyclopentadiene, 2-methyl-1,3-cyclohectadiene, 2,3-dimethyl-1,3-cyclopentadiene, 2,3-dimethyl-1,3-cyclohexadiene, 1,3-cyclohexadiene, 2-chloro-1,3-butadiene, 2,3-dichloro-1,3-butadiene, 1-fluoro-1,3-butadiene, 2-chloro-1,3-pentadiene, 2-chloro-1,3-cyclopentadiene and 2-chloro-1,3-cyclohexadiene. In some embodiments, isoprene, polyisoprene or isoprene derivatives or polyisoprene derivatives may also be used in the adhesive. If desired, the adhesive may be a pressure sensitive adhesive. In certain examples, the adhesive can be crosslinkable to the substrate using light, heat, a catalyst, an activator or other suitable materials and/or processes.

In certain configurations, an adhesive layer can be present between any two or more component or layers of the articles. For example, an adhesive layer can be present between the carrier support material and the underlying substrate, between the carrier support material and any bioactive surface coating or between other layers that may be present in the articles.

In some examples, a release liner may be present on a surface of the article that is to be coupled to an underlying article or device. The release liner is typically an inert material, e.g., a paper, plastic, rubber, etc. that is used to cover the adhesive layer prior to use of the article. The release liner may comprise, for example, Kraft paper, clay coated paper, machine glazed paper, a polyethyleneterephthalate film, a polyethylene film, a polypropylene film, and other films produced using polyolefin materials. In use of the article, the release liner is typically peeled away from the article to expose the adhesive layer, and the article is pressed onto a desired surface and retained on the surface through the adhesive layer. The article can then be removed at a later time by mechanical force, using heat or a solvent or through other means.

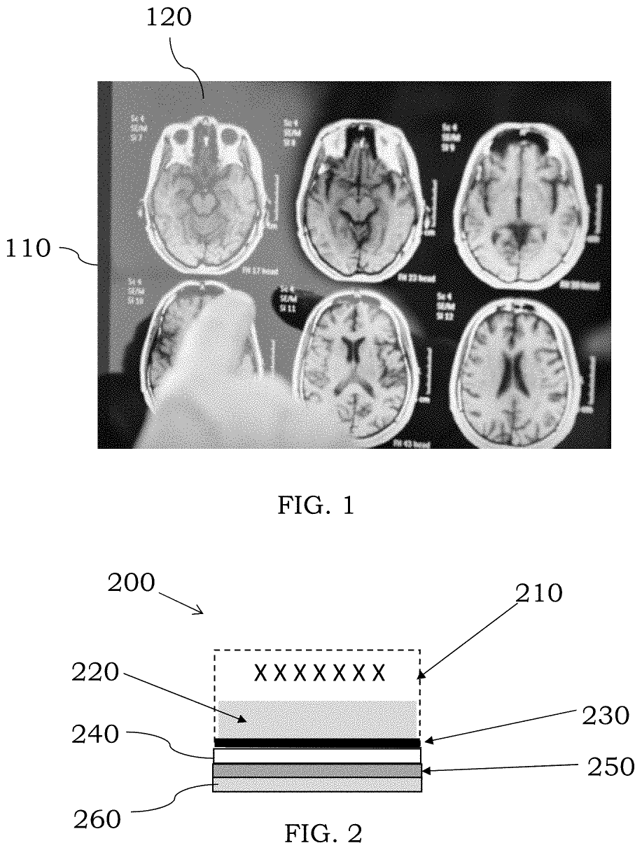

In certain configurations, an illustration showing an electronic device screen cover coupled to a front surface of an electronic device is shown in FIG. 1. While not shown, the electronic device screen 110 can be placed in a case and the screen covers would then be attached to the case. The screen cover 120 is attached to a front surface of the electronic device screen 110 through an adhesive layer on the screen cover 120. While the screen cover 120 is shown as a single cover that spans across the entire screen 110, smaller screen covers could be applied beside each other if desired. For example, three, four, five, six or more screen covers can be present. In addition, the screen cover can be configured as a stack to permit a user to tear off a top screen an expose an underlying new screen if desired.

Referring to FIG. 2, one illustration of certain components and materials that are present in a screen cover is shown. The screen cover 200 comprises a surface coating 210, a carrier support material 220 configured as a dome, an adhesive layer 230, a substrate 240, another adhesive layer 250 and a release liner 260. In this illustration, the bioactive material is present in the surface coating 210, which can be sprayed, dip coated, curtain coated, roller coated, printed, brushed or otherwise deposited on the carrier support material 220. If desired, two or more layers of the surface coating 210 can be sprayed onto the support material 220 as individual layers. The carrier support material 220 can be any of those materials mentioned above or other suitable materials. The adhesive layer 230 acts to retain the carrier support material 220 to the underlying substrate 240. The adhesive layer 250 retains the article 200 to the surface of the electronic device screen when in use. The adhesive layers 230, 250 can comprise the same or different materials as desired and may be any of the illustrative adhesive materials mentioned herein or other suitable adhesive materials. The substrate 240 is typically optically transparent and may be any of those illustrative materials discussed herein. While certain layers are shown in FIG. 2 as comprising the same thickness, this arrangement is not required or even typical. The adhesive layers 230, 250 tend to be much thinner than the substrate 240 or the carrier support material 220. The bioactive material in the surface coating 210 typically comprises one or more transition metals or transition metal materials including, but not limited to, those comprising titanium, zinc, copper, silver or other transition metals mentioned herein. The bioactive material in the surface coating 210 may be present in ionic form, chelated or bound to other groups or both. In some instances, the bioactive material of the surface coating 210 can be photo-activated by exposure to ultraviolet light or visible light (or both) and can be photo-recharged upon re-exposure to ultraviolet light or visible light (or both). In other examples, the bioactive material in the surface coating 210 can function as a photocatalyst to kill or inactivate bioorganisms that contact the surface coating 210.

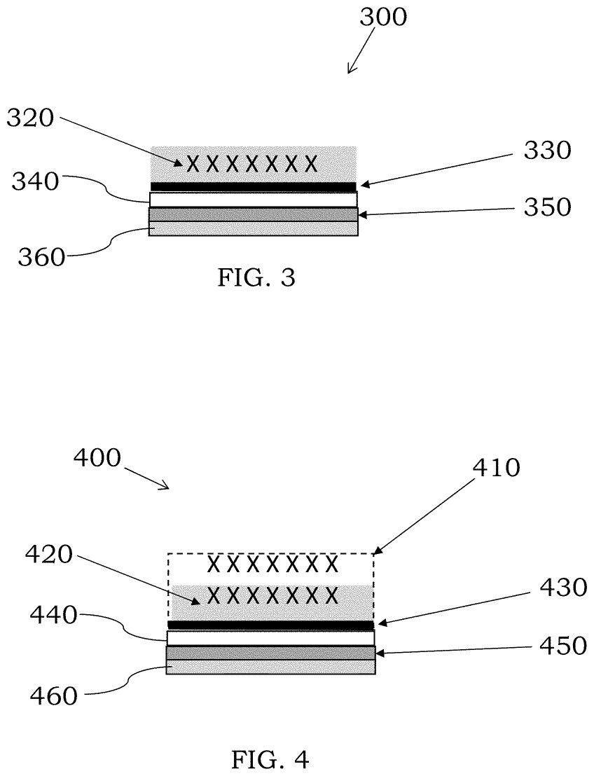

In certain configurations, other material arrangements for the electronic device screen covers are also possible. Referring to FIG. 3, another illustration of certain components and materials that are present in a screen cover is shown. The screen cover 300 comprises a surface coating 320 comprising a carrier support material and a bioactive material, an adhesive layer 330, a substrate 340, another adhesive layer 350 and a release liner 360. In this illustration, the bioactive material is embedded within the carrier support material that is present in a surface coating 320, which can be sprayed, dip coated, curtain coated, roller coated, printed, brushed or otherwise deposited on the underlying adhesive layer 330. The carrier support material in the coating 320 can be any of those materials mentioned above or other suitable materials. The adhesive layer 330 acts to retain the carrier support material 320 to the underlying substrate 340. The adhesive layer 350 retains the article 300 to the surface of the electronic device screen when in use. The adhesive layers 330, 350 can comprise the same or different materials as desired and may be any of the illustrative adhesive materials mentioned herein or other suitable adhesive materials. The substrate 340 is typically optically transparent and may be any of those illustrative materials discussed herein. While certain layers are shown in FIG. 3 as comprising the same thickness, this arrangement is not required or even typical. The adhesive layers 330, 350 tend to be much thinner than the substrate 340 or the surface coating 320. The bioactive material embedded in the carrier support material present in the surface coating 320 typically comprises one or more transition metals or transition metal materials including, but not limited to, those comprising titanium, zinc, copper, silver or other transition metals mentioned herein. The bioactive material in the surface coating 320 may be present in ionic form, chelated or bound to other groups or both. In some instances, the bioactive material of the surface coating 320 can be photo-activated by exposure to ultraviolet light or visible light (or both) and can be photo-recharged upon re-exposure to ultraviolet light or visible light (or both). In some examples, the bioactive material in the surface coating 320 can function as a photocatalyst to kill or inactivate bioorganisms that contact the surface coating 320.

Referring to FIG. 4, an illustration is shown where a bioactive material is present in both a surface coating and is embedded in a carrier support material that underlies the surface coating. The screen cover 400 comprises a surface coating 410 with a bioactive material, a carrier support material 420 (with a bioactive material), an adhesive layer 430, a substrate 440, another adhesive layer 450 and a release liner 460. In this illustration, the bioactive material is present in the surface coating 410, which can be sprayed, dip coated, curtain coated, roller coated, printed, brushed or otherwise deposited on the carrier support material 420. In addition, the carrier support material 420 also comprises an embedded bioactive material which may be the same or may be different than the bioactive material of the surface coating 410. The carrier support material 420 can be any of those materials mentioned above or other suitable materials. The adhesive layer 430 acts to retain the carrier support material 420 to the underlying substrate 440. The adhesive layer 450 retains the article 400 to the surface of the electronic device screen when in use. The adhesive layers 430, 450 can comprise the same or different materials as desired and may be any of the illustrative adhesive materials mentioned herein or other suitable adhesive materials. The substrate 440 is typically optically transparent and may be any of those illustrative materials discussed herein. While certain layers are shown in FIG. 4 as comprising the same thickness, this arrangement is not required or even typical. The adhesive layers 430, 450 tend to be much thinner than the substrate 440 or the carrier support material 420. The bioactive material in the surface coating 410 and in the carrier support material 420 typically comprises one or more transition metals or transition metal materials including, but not limited to, those comprising titanium, zinc, copper, silver or other transition metals mentioned herein. The bioactive material in the surface coating 410 and in the carrier support material 420 may be present in ionic form, chelated or bound to other groups or both. In some instances, the bioactive material of the surface coating 410 and the carrier support material 420 can be photo-activated by exposure to ultraviolet light or visible light (or both) and can be photo-recharged upon re-exposure to ultraviolet light or visible light (or both). In some instances, the bioactive material in the surface coating 410 and in the carrier support material 420 can independently function as a photocatalyst to kill or inactivate bioorganisms that contact the screen cover 400.

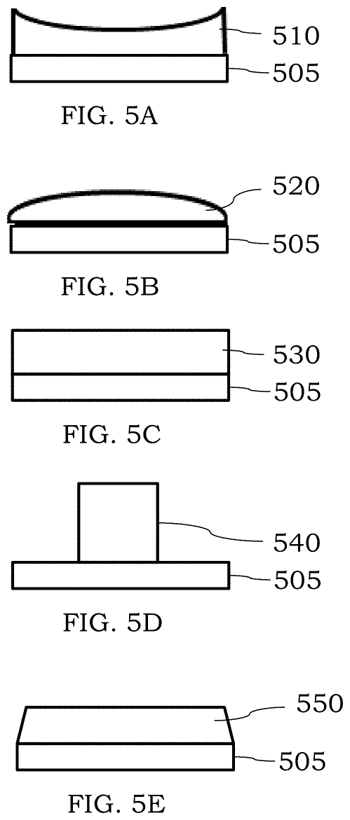

While the carrier support material is shown in FIG. 2-4 as being substantially flat, this shape is not required. For example, a concave shape 510 (FIG. 5A), an half-ellipse shape 520 (FIG. 5B), a rectangular shape 530 (FIG. 5C), a square shape 540 (FIG. 5D), a trapezoidal shape 550 (FIG. 5E) or other geometric shapes for the carrier support layer present on a substrate 505 could instead be present and used to produce the articles described herein. In addition, the carrier support material need not comprise a uniform thickness across the surface of the screen cover.

In some examples, a plurality of electronic device screen covers can be coupled to a single release liner. For example, where the screen cover is sized and arranged to cover a cellular phone screen, more than a single screen cover can be present on the release liner. Alternatively, two more screen covers can be applied to a single display screen at different areas. The screen covers can be the same or can be different, e.g., may comprise different materials and/or different shapes. In addition, the exact number of screen covers used with an electronic device screen may vary from one to more than six as desired.

In other examples, the screen covers described herein may be permanently attached to an electronic device screen or may be integral to the screen. For example, an electronic device screen may comprise a permanently attached or integral electronic device screen cover or covers. For example, the permanently attached or integral electronic device screen cover may comprise a bioactive material comprising at least one of titanium, silver, copper and zinc or other transition metals and combinations thereof. In other examples, the bioactive material can be photo-rechargeable. The carrier support material may comprise a polyurethane. If desired, the screen cover may comprise a second bioactive material embedded in the polyurethane of the carrier support material. In some examples, the bioactive material and the second bioactive material comprise the same material or different transition metals. In some embodiments, the screen cover may comprise a first adhesive layer comprising a residue free adhesive. The carrier support material may comprise a dome shape or other suitable shapes. The integral or permanently attached case screen cover can be optically transparent. Where a permanent screen cover is attached to the electronic device screen, the substrate may comprise a polyolefin. In some examples, the bioactive material of the permanently attached or integral screen cover can function as a photocatalyst, e.g., may comprise photocatalytic titanium dioxide or similar photocatalytic transition metal materials

In other configurations, the permanently attached or integral electronic device screen cover may comprise a first adhesive layer coupled to the electronic device screen, a substrate coupled to the first adhesive layer, a second adhesive layer coupled to the substrate, and a surface coating coupled to the second adhesive layer, wherein the surface coating comprises a carrier support material with an embedded bioactive material to inactivate or kill bioorganisms that contact the surface coating. In some examples, the bioactive material comprises at least one of titanium, silver, copper and zinc. In certain examples, the bioactive material is photo-rechargeable. In other examples, the carrier support material comprises a polyurethane. In some embodiments, the first adhesive layer comprises a residue free adhesive. In other embodiments, the carrier support material is substantially planar. In additional examples, the electronic device screen cover is optically transparent. In certain examples, the substrate comprises a polyolefin. In other examples, the substrate comprises a polyethylene, the carrier support material comprises a polyurethane, and the embedded bioactive material comprises a transition metal and is photo-rechargeable. In some examples, the screen cover further comprises an additional bioactive material disposed on a surface of the carrier support material. In some examples, the bioactive material of the screen covers can function as a photocatalyst.

In other configurations, an electronic device comprises an integral electronic device screen cover. For example, the electronic device screen comprises a front surface configured to receive the integral electronic device screen cover. In some instances, the integral electronic device screen cover comprises a substrate coupled to the electronic device screen, a first adhesive layer coupled to the substrate, a carrier support material coupled to the first adhesive layer, and a surface coating coupled to the carrier support material, the surface coating comprising a bioactive material to inactivate or kill bioorganisms that contact the surface coating. In some examples, the carrier support material comprises a dome shape. In certain embodiments, the electronic device screen cover is optically transparent. In some embodiments, the substrate comprises a polyolefin and the carrier support material comprises a polyurethane. In certain examples, the bioactive material comprises at least one of titanium, silver, copper and zinc. In some examples, the bioactive material can function as a photocatalyst.

In additional configurations, an electronic device screen comprises an integral electronic device screen cover comprising a substrate coupled to the electronic device screen, a first adhesive layer coupled to the substrate, and a surface coating coupled to the first adhesive layer, wherein the surface coating comprises a carrier support material with an embedded bioactive material to inactivate or kill bioorganisms that contact the surface coating. In some embodiments, the carrier support material comprises a substantially planar shape. In other examples, the integral electronic device screen cover is optically transparent. In certain examples, the substrate comprises a polyolefin and the carrier support material comprises a polyurethane. In some examples, the bioactive material comprises at least one of titanium, silver, copper and zinc. In other examples, the bioactive material can function as a photocatalyst.

In other examples, an electronic device screen comprises an integral electronic device screen cover comprising a carrier support material coupled to the electronic device screen, and a surface coating coupled to the carrier support material, the surface coating comprising a bioactive material to inactivate or kill bioorganisms that contact the surface coating. In some examples, the carrier support material comprises a substantially planar shape. In other examples, the integral electronic device screen cover is optically transparent. In additional examples, the substrate comprises a polyolefin and the carrier support material comprises a polyurethane. In other examples, the bioactive material comprises at least one of titanium, silver, copper and zinc. In some examples, the bioactive material can function as a photocatalyst.

In other embodiments, an electronic device screen comprises an integral electronic device screen cover comprises a surface coating coupled to the electronic device screen, wherein the surface coating comprises a carrier support material with an embedded bioactive material to inactivate or kill bioorganisms that contact the surface coating. In some examples, the carrier support material comprises a substantially planar shape. In other examples, the integral electronic device screen cover is optically transparent. In additional examples, the substrate comprises a polyolefin and the carrier support material comprises a polyurethane. In some examples, the bioactive material comprises at least one of titanium, silver, copper and zinc. In some examples, the bioactive material can function as a photocatalyst.

The articles and various layers described herein can also be used with additional materials including primers, e.g., titanium dioxide primer layers, colorants, inks, luminescent coatings, surfactants and other materials as desired. Crosslinkers such as amides or other materials can also be used to facilitate rapid curing of the layers or the layers can be cured without the use of any crosslinkers. In some embodiments, one or more of a halogenated phenol, a phenoxy phenol, a hydroxyphenyl ether, a halogenated phenoxy, e.g., fluorinated, chlorinated or brominated phenoxy compounds, polyhexamethylene biguanide (PHMD), PHMD chloride, PHMD fluoride, PHMD bromide, PHMD hydrochloride, Microban® materials, halogenated phenols such as, for example, 5-chloro-2-(2,4-dichlorophenoxy) phenol, chloro-2-(2,4-dichloro)phenol, and chloro-2-(2,4-dichlorophenoxy)phenol, Triclosan, Irgansan DP300, CH3635, Ster-zac, Lexol 300, trichloro-2-hydroxydiphenyl ether, plant oils such as, for example, tea tree oil, mint oil, leleshwa oil, sandalwood oil, clove oil, lavender oil, nigella sativa (Black cumin) oil, onion, garlic and combinations thereof can also be present in the surface coating or carrier support material or both. In some instances, the surface coating, carrier support material or both may include one or more materials commercially available from Environ (Rochester Hills, Mich.), Microban (Huntersville, N.C.), or Oxititan (Pompano Beach, Fla.) or other producers of antimicrobial ingredients.

In some embodiments, the bioactive materials described herein can be also present in a housing of an electronic device screen or on the electronic screen itself. For example, the screen may comprise a surface coating comprising a bioactive material to inactivate or kill bioorganisms that contact the surface coating. In other instances, the screen may also comprise a carrier support material and a surface coating coupled to the carrier support material, the surface coating comprising a bioactive material to inactivate or kill bioorganisms that contact the surface coating. The screen is typically an LCD, LED, OLED or other electronic screens that are commonly used, for example, in cellular phones such as Iphone® devices sold commercially by Apple or Galaxy® devices sold commercially by Samsung. The electronic device screen can also be present in a tablet, laptop, portable television or monitor, a seatback or headrest display on aircraft or other vehicles or other electronic devices such as those found in a kiosk. The electronic device typically comprises a processor and one or more memory units that can be used together to display application software or other desired information or programs.

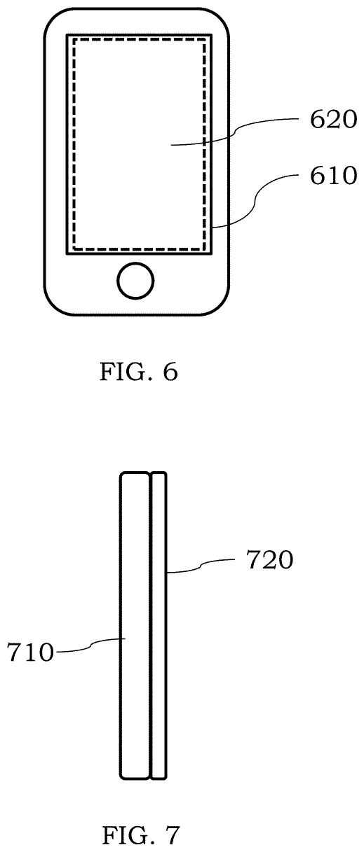

Referring to FIG. 6, an illustration of a mobile device 610 that comprises a screen cover 620 is shown. The screen cover 620 may be any of those described herein. The mobile device 610 may be a cellular phone or other portable mobile devices that comprise a display screen. The screen cover 620 may comprise one or more of a first adhesive layer coupled to the display screen of the mobile device 610, a substrate coupled to the first adhesive layer, a second adhesive layer coupled to the substrate a carrier support material coupled to the second adhesive layer, and/or a surface coating coupled to the carrier support material, the surface coating comprising a bioactive material to inactivate or kill bioorganisms that contact the surface coating.

Referring to FIG. 7, a side view of an electronic device screen 710 that comprises a coating 720 comprising a bioactive material is shown. For example, the coating 720 can comprise a bioactive material to inactivate or kill bioorganisms that contact the surface coating. In other examples, the coating 720 may comprise a carrier support material and a surface coating coupled to the carrier support material, wherein the surface coating comprises a bioactive material to inactivate or kill bioorganisms that contact the surface coating.

Certain embodiments described herein can be used in methods and device to reduce infections and community spread of infections. The methods can desirably use one or more of the screen covers or screens described herein.

In some embodiments, a method of reducing infections comprises placing one or more of the screen cover or screens described herein onto another article or device to facilitate transfer of infectious organisms, infectious virus, infectious viral agents or infectious viral particles from a user to the screen cover or screen. A bioactive material on the placed screen cover or screen can inactivate and/or kill the infectious virus, infectious viral agents or infectious viral particles to prevent infection of a subsequent user who contacts the screen cover or screen.

In another embodiment, a method of reducing community spread of an infection comprises placing one or more of the screen cover or screens described herein onto another article or device facilitate transfer of infectious organisms, infectious virus, infectious viral agents or infectious viral particles from a user to the screen cover or screen. A bioactive material on the placed screen cover or screen can inactivate and/or kill the infectious virus, infectious viral agents or infectious viral particles to prevent community spread of the infectious virus, infectious viral agents or infectious viral particles.

In another example, a method of treating a person infected with an infection while reducing spread of the infection from the infected person comprises placing a screen cover or screen comprising one or more bioactive materials on a corresponding receptive article and administering to the infected person in need of treatment one or more antiviral drugs, antimicrobial drugs or anti-parasitic drugs or combinations thereof. The drug administration can treat the infected person while the placed screen cover or screen can reduce spread of the infection from the human being treated to third parties. For example, the method can reduce spread by killing or inactivating of infectious organisms, infectious virus, infectious viral agents or infectious viral particles that have been transferred to the article using one or more bioactive materials on the screen cover or screen.

In certain examples, the methods and screen covers or screens described herein can be used to prevent or reduce the spread of a virus including double-stranded DNA viruses, a single-stranded DNA virus, a double-stranded RNA virus, a single stranded RNA virus, a single-stranded RNA retrovirus, a double- stranded DNA retrovirus and other viruses including either double-stranded DNA or RNA or single stranded DNA or RNA or hybrid DNA-RNA nucleic acid. Specific types of viruses include, but are not limited to, a picornavirus, a coronavirus, a rhinovirus, an adenovirus, an enterovirus, an influenza virus, a human parainfluenza virus, a human respiratory syncytial virus, a metapneumovirus, a retrovirus, a norovirus, a rotavirus, a herpes virus, a poxvirus, a reovirus, an orthomyxovirus, a rhabdovirus, a parvovirus and other viruses that can infect mammals such as humans or other animals. As noted in more detail below, the devices are particularly effective at reducing active levels of coronaviruses such as, for example, coronavirus 229E, coronavirus NL63, coronavirus 0C43, coronavirus HKU1, MERS-CoV, SARS-CoV and SARS-CoV-2 (COVID-19). In some instances, at least 95% of the coronavirus transferred to the surface can be killed or inactivated by the bioactive material within 30 minutes after transfer to the surface. In other instances, at least 95% of the coronavirus transferred to the surface can be killed or inactivated by the bioactive material within 60 minutes after transfer to the surface. In some embodiments, at least 95% of the coronavirus transferred to the surface can be killed or inactivated by the bioactive material within 120 minutes after transfer to the surface.

In some instances, at least 99% of the coronavirus transferred to the screen cover or screen surface can be killed or inactivated by the bioactive material within 30 minutes after transfer to the surface. In other instances, at least 99% of the coronavirus transferred to the screen cover or screen surface can be killed or inactivated by the bioactive material within 60 minutes after transfer to the surface. In some embodiments, at least 99% of the coronavirus transferred to the screen cover or screen surface can be killed or inactivated by the bioactive material within 120 minutes after transfer to the surface.

In certain embodiments, the methods and screen covers or screens described herein can be used to prevent or reduce the spread of infections from one or more bacteria, including but not limited to, Bacillus, Pseudomonas, Bacteroides, Bordetella, Brucella, Campylobacter, Chlamydia, Clostridium, e.g., Clostridium difficile, Corynebacterium, Enterobacter, Enterococcus, Escherichia, Haemophilus, Klebsiella, Lactobacillus, Legionella, Listeria, Micrococcus, Mycobacterium, Mycoplasma, Neisseria, Rickettsia, Salmonella, Shigella, Staphylococcus, Streptococcus, Vibrio, Yersinia and other bacteria commonly encountered in a clinical setting. In certain examples, spores such as those from Bacillus species or Clostridium species, e.g., Clostridium difficile, can be inactivated. In some embodiments, the bioactive material can be effective to kill or inactivate one or more of Actinobacteria, Bacteriodetes, Firmicutes, Propionibacteriaceae, Lactobacillaceae and Proteobacteria as these bacteria are commonly encountered in public setting such as public restrooms and surfaces therein. If desired, the bioactive material can also be selected to kill or inactivate fungal organisms such as those commonly encountered in athletic facility showers, e.g., Tinea, Trichophyton, Candida and other fungal organisms.

In certain embodiments, any of the screen covers or screens described herein can be printed by applying suitable materials to a surface using a printer. The printer may be, for example, an inkjet printer, digital printer, laser printer, etc.

In certain instances, the screen covers or screen described herein can be used in combination with an antimicrobial agent or therapeutic. Illustrative antimicrobial agents include, but are not limited to, a sulfonamide, a trimethoprim-sulfamethoxazole, a quinolone, a fluoroquinolone, a quinone, a penicillin, a cephalosporin, a Beta-lactam antibiotic, a Beta-lactamase inhibitor, an aminoglycoside, a tetracycline, a chloramphenicol, an erythromycin, a macrolide, a clindamycin, isoniazid, rifampin, a pyrazinamide, an ethionamide, amphotericin B, imidazole, triazole, ketoconazole, miconazole, itraconazole, fluconazole, ciclopirox olamine, haloprogin, tolnaftate, naftifine, terbinafine, chloroquinone, and hydroxychloroquinone. Other antibacterial and antifungal agents could also be used. Combinations of two or more of any of these antimicrobial agents can also be used in combination with the screen cover or screen described herein.

In some embodiments, the screen covers or screen described herein can be used in combination with one or more antiviral agents or therapeutics including, but not limited to, Abacavir, Acyclovir, Adefovir, Amantadine, Ampligen, Amprenavir, Arbidol, Atazanavir, Atripla, Balavir, Baloxavir marboxil, Biktarvy, Boceprevir, Cidofovir, Cobicistat, Combivir, Daclatasvir, Darunavir, Delavirdine, Descovy, Didanosine, Docosanol, Dolutegravir, Doravirine, Ecoliever, Edoxudine, Efavirenz, Elvitegravir, Emtricitabine, Enfuvirtide, Entecavir, Etravirine, Famciclovir, Fomivirsen, Fosamprenavir, Foscarnet, Fosfonet, a fusion inhibitor, Ganciclovir (Cytovene), Ibacitabine, Ibalizumab (Trogarzo), Idoxuridine, Imiquimod, Imunovir, Indinavir, Inosine, Integrase inhibitor, Interferon type I, Interferon type II, Interferon type III, Interferon, Lamivudine, Letermovir (Prevymis), Lopinavir, Loviride, Maraviroc, Methisazone, Moroxydine, Nelfinavir, Nevirapine, Nexavir, Nitazoxanide, Norvir, Nucleoside analogues, Oseltamivir (Tamiflu), Peginterferon alfa-2a, Peginterferon alfa-2b, Penciclovir, Peramivir (Rapivab), Pleconaril, Podophyllotoxin, a protease inhibitor, Pyramidine, Raltegravir, Remdesivir, a reverse transcriptase inhibitor, Ribavirin, Rilpivirine (Edurant), Rimantadine, Ritonavir, Saquinavir, Simeprevir (Olysio), Sofosbuvir, Stavudine, Synergistic enhancer (antiretroviral), Telaprevir, Telbivudine (Tyzeka), Tenofovir alafenamide, Tenofovir disoproxil, Tenofovir, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), Zidovudine and combinations thereof.

In some embodiments, an antimicrobial agent can be used in combination with an antiviral agent and one or more of the screen covers or screens described herein. For example, the antiviral can be used to treat a viral infection, an antimicrobial can be used to treat a secondary bacterial infection and the screen covers or screens described herein can be used to prevent or reduce spread of the virus and/or antimicrobials to third parties.

In some embodiments, the screen covers or screens described herein can be dispensed in a vending machine or other devices to permit addition of the screens cover or screens to another device by an end user. For example, the screen cover or screens can be placed in public places such as offices, banks, grocery stores, train stations, subway stations, airports, etc. to permit a user to purchase and place the screen cover or screens on an article such as a laptop, tablet, mobile device or other electronic or non-electronic device.

Certain specific examples are described showing the articles and materials thereon can be used to kill or inactivate viruses and bioorganisms.

### Example 1

A 8.5 inches by 11 inches sheet of material including a bioactive material comprising titanium dioxide doped with silver ions present in a surface coating was aseptically cut into 1″×1″ squares. Stainless steel control squares of the same size were ethanol sanitized and double rinsed in reverse osmosis prepared water and then autoclaved prior to use. Each of the test and control samples were placed into sterile Petri dishes using sterile forceps.

A stock vial of human coronavirus 229E (ATCC VR-740) was removed from cryo-storage and permitted to thaw. 0.010 mL aliquots were aseptically spread over the surface of each test and control square to ˜⅛ inch of the edge. Virus films were prepared in duplicate per test and control surface, per contact time (T=30 min, 1 hour, 2 hours and 4 hours). Control and test carrier were dried with Petri dish lids slightly ajar for 20 minutes at 24.7 degree Celsius, 36% relative humidity, Illuminance 1140 lux. Contact times were initiated when the control and test squares were visibly dry.

At the end of each contact time, the test and control carriers were aseptically transferred to tubes containing 2.0 mL of neutralizing solution (2% FBS EMEM). The carriers were vortexed for 30 seconds each to mechanically dislodged the microorganisms for enumeration. The inoculated sides of each carrier were further treater using a cell scraper to ensure adequate removal of the test viruses.

For cytotoxicity and neutralization effectiveness controls, one test and one control carrier each (with no virus film) were each aseptically transferred to neutralization tubes, and vortexed as described previously for the virus. The vortexed suspensions were serially diluted ten-fold in neutralizing solution, and selected dilutions were plated in quadruplicate onto the appropriate host cell monolayers (MRC-5, ATCC CCL-171) prepared to suitable confluency in multi-well trays. Virus control, cytotoxicity, neutralization validation, and sterility controls were performed concurrently. Virus reductions were calculated using the Spearman-Karber Method. Reference may be made to JIS Z 2801:2000. Antimicrobial Products—Test for Antimicrobial Activity and Efficacy. Japanese Standards Association. Tokyo, Japan.

No cytotoxicity was observed for the MRC-5 cells on the stainless steel control and tested squares.

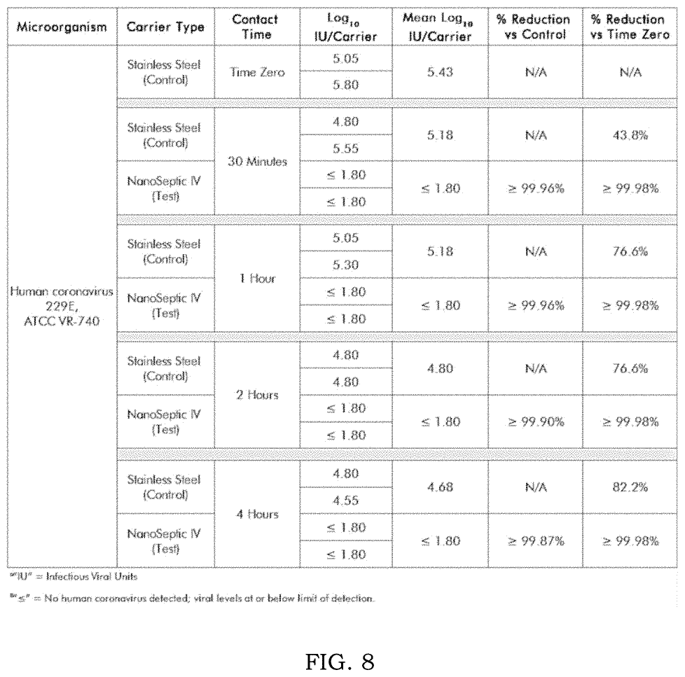

Referring to FIG. 8, as can be seen the test samples (labeled Nanoseptic IV), showed over a 99.96% reduction in viral for all measured times. In contrast, stainless steel control samples showed significantly less reduction at all measured times. These results are consistent with the tested samples being able to inactive the coronavirus and prevent infection of the MRC-5 human lung fibroblast cells.

### Example 2

An article (2 inches by 2 inches) comprising a bioactive material comprising titanium dioxide doped with silver ions in a surface coating was tested for its ability to kill E. coli. using a modified ISO 22196 protocol. An overnight culture of E. coli cells (ATCC 8739) was diluted in sterile 1:500 Nutrient Broth. A sterile swab was dipped into the prepared test inoculum and used to inoculate each carrier via 13 passes (left to right =1 pass). Inoculated carriers were allowed to dry for 5 minutes followed by initiation of the contact time. Carriers were harvested after 5, 20, 60 and 120 minutes, vortexed to elute the viable bacteria and enumerated using standard dilution and pour plate techniques. Three replicates at each contact time were measured. Percent reduction was calculated as 100×(C−A)/C where A was the number of bacteria on the test carriers after the contact time and C is the number of bacteria on the control at time zero.

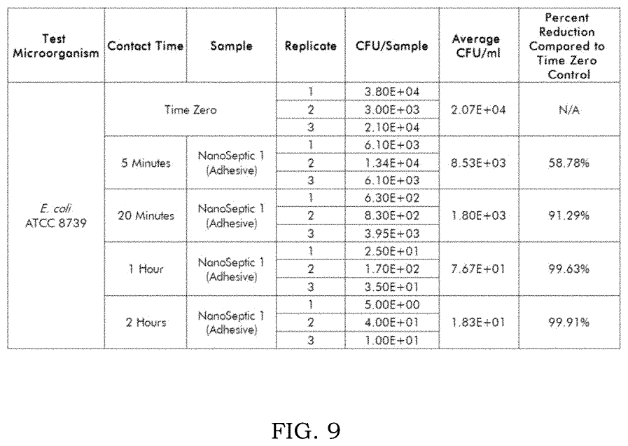

The results are shown in FIG. 9. At 20 minutes, a reduction in over 90% was observed. At 1 hour a reduction over 99% was observed. These results are consistent with the tested samples being able to kill the E. coli.

When introducing elements of the aspects, embodiments and examples disclosed herein, the articles “a,” “an,” “the” and “said” are intended to mean that there are one or more of the elements. The terms “comprising,” “including” and “having” are intended to be open-ended and mean that there may be additional elements other than the listed elements. It will be recognized by the person of ordinary skill in the art, given the benefit of this disclosure, that various components of the examples can be interchanged or substituted with various components in other examples.

Although certain aspects, examples and embodiments have been described above, it will be recognized by the person of ordinary skill in the art, given the benefit of this disclosure, that additions, substitutions, modifications, and alterations.

1. An electronic device screen cover configured to couple to an electronic device screen, the electronic device screen cover comprising:
a release liner;
a first adhesive layer coupled to the release liner;
a substrate coupled to the first adhesive layer;
a second adhesive layer coupled to the substrate;
a carrier support material coupled to the second adhesive layer; and
a surface coating coupled to the carrier support material, the surface coating comprising a bioactive material to inactivate or kill bioorganisms that contact the surface coating.

2. The electronic device screen cover of claim 1, wherein the bioactive material comprises at least one of titanium, silver, copper and zinc.

3. The electronic device screen cover of claim 2, wherein the carrier support material comprises a polyurethane.

4. The electronic device screen cover of claim 1, wherein the bioactive material comprises a photocatalyst comprising titanium, and wherein the bioactive material comprises at least one additional transition metal.

5. The electronic device screen cover of claim 3, further comprising a second bioactive material embedded in the polyurethane of the carrier support material.

6. The electronic device screen cover of claim 5, wherein the bioactive material and the second bioactive material comprise different transition metals.

7. The electronic device screen cover of claim 3, wherein the first adhesive layer comprises a residue free adhesive.

8. The electronic device screen cover of claim 3, wherein the carrier support material comprises a substantially planar shape.

9. The electronic device screen cover of claim 8, wherein the electronic device screen cover is optically transparent.

10. The electronic device screen cover of claim 9, wherein the substrate comprises a polyolefin.

11. An electronic device screen cover configured to couple to an electronic device screen, the electronic device screen cover comprising:
a release liner;
a first adhesive layer coupled to the release liner;
a substrate coupled to the first adhesive layer;
a second adhesive layer coupled to the substrate; and
a carrier support material coupled to the second adhesive layer, wherein the carrier support material comprises an embedded bioactive material to inactivate or kill bioorganisms that contact the carrier support material.

12. The electronic device screen cover of claim 11, wherein the bioactive material comprises at least one of titanium, silver, copper and zinc.

13. The electronic device screen cover of claim 12, wherein the carrier support material comprises a polyurethane.

14. The electronic device screen cover of claim 11, wherein the bioactive material comprises a photocatalyst comprising titanium, and wherein the bioactive material comprises at least one additional transition metal.

15. The electronic device screen cover of claim 13, further comprising a second bioactive material embedded in the polyurethane of the carrier support material.

16. The electronic device screen cover of claim 15, wherein the bioactive material and the second bioactive material comprise different transition metals.

17. The electronic device screen cover of claim 13, wherein the first adhesive layer comprises a residue free adhesive.

18. The electronic device screen cover of claim 13, wherein the carrier support material comprises a substantially planar shape.

19. The electronic device screen cover of claim 18, wherein the electronic device screen cover is optically transparent.

20. The electronic device screen cover of claim 19, wherein the substrate comprises a polyolefin.

21-67. (canceled)
